(19) United States
(12) Patent Application Publication
(10) Pub. No.: US 2026/0232369 A1
(43) Pub. Date: Aug. 13, 2026

(54) Power Generator for Electrosurgical Use

(71) Applicant: AMX Technologies, LLC, Plymouth, MN (US)

(72) Inventors: Paul Sorajja, Plymouth, MN (US); Eric N. Rudie, Plymouth, MN (US); Daniel P. Coyle, Plymouth, MN (US); David M. Costello, Plymouth, MN (US)

(73) Assignee: AMX Technologies, LLC, Plymouth, MN (US)

(21) Appl. No.: 19/161,192

(22) PCT Filed: Mar. 7, 2024
(86) PCT No.: PCT/US2024/018911

§ 371 Date: Aug. 29, 2025

Related U.S. Application Data
(60) Provisional application No. 63/488,950, filed on Mar. 7, 2023.

(51) Int. Cl. A61B 18/12; A61B 17/00; A61B 18/00

(52) U.S. Cl. CPC A61B 18/1206; A61B 2017/00172; A61B 2017/00199; A61B 2018/00601; A61B 2018/00642; A61B 2018/00678; A61B 2018/00702; A61B 2018/00726; A61B 2018/00767; A61B 2018/00839; A61B 2018/00875; A61B 2018/00886

(57) ABSTRACT

The present invention discloses an RF electrosurgical system featuring an RF electrosurgical generator with a user interface and outlets delivering RF energy to an RF cutting instrument. The RF electrosurgical generator is equipped with software designed to measure impedance values while delivering voltage pulses to the cutting instrument. In the event that the measured impedance value surpasses a predetermined impedance threshold, the software is programmed to adjust the voltage amplitude of each voltage pulse, either partially or fully reducing it. This innovative system ensures optimal performance and safety during electrosurgical procedures by dynamically adapting voltage output based on real-time impedance measurements.

### RELATED APPLICATIONS

This application claims benefit of and priority to U.S. Provisional Application Ser. No. 63/488,950 filed Mar. 7, 2023 entitled Generator for Electrosurgical Use, which is hereby incorporated herein by reference in its entirety.

### BACKGROUND OF THE INVENTION

Electrosurgery involves the application of high-frequency alternating polarity, electrical current (radiofrequency current or RF current) to biological tissue as a means to cut, coagulate, desiccate, or fulgurate tissue. Its benefits include the ability to make precise cuts with limited blood loss.

The RF current or energy typically alternates between about 100 KHz to about 4 MHz to induce ionic vibrations that generate intracellular heat. Depending on several aspects of the current, different levels of intracellular heat may be quickly reached to produce different results with the tissue, specifically, cutting, coagulation, desiccation, and fulguration. There are typically wet and dry field electrosurgical devices. Wet field devices operate in a saline solution or in bodily fluid as a result of an alternating current that passes between two electrodes or a single electrode and a ground return pad.

Most wet field electrosurgical systems typically at least have a cutting mode and a coagulation mode. A cutting mode may cause a small area of tissue to quickly increase in temperature and create a vapor pocket which vaporizes and explodes a small section of soft tissue, resulting in an incision. Peak voltage may be very high but the average power needed for cutting tissue is relatively low. In a coagulation mode, the average voltage output is typically higher than in cutting mode so that more thermal heating is produced and the tissue remains grossly intact, but cells are destroyed at the point of contact and smaller vessels are destroyed and sealed, stopping capillary and small-arterial bleeding. Fulguration modes are typically used with dry field electrosurgical procedures and create an electric arc discharge between an air gap and the tissue to a more superficial “burning” of the tissue. However, arc discharges may also be possible during wet field electrosurgical procedures, either intentionally or unintentionally.

RF electrosurgery is typically performed using an RF electrosurgical generator and a surgical instrument that includes either one electrode (a monopolar instrument) or two electrodes (a bipolar instrument). Some RF electrosurgical generators are able to be used with either monopolar instruments or bipolar instruments.

Typically, monopolar instruments have a single electrode or conductive surface connected to the RF electrosurgical generator while a second electrode, often in the form of a pad or external adhesive electrode, is placed in contact with a patient's skin to complete the circuit of the RF electrosurgical generator. A bipolar instrument often includes two electrodes connected to different circuit paths which allow the electrical circuit of the RF electrosurgical generator to be completed as they are placed near or in contact with tissue. In wet field procedures, monopolar instruments often are more successful for vaporizing or cutting tissue with bipolar instruments.

### SUMMARY OF THE INVENTION

In some aspects, the techniques described herein relate to an RF electrosurgical system, including: an RF electrosurgical generator including a user interface and one or more outlets supplying RF energy to an RF cutting instrument connected thereto; the RF electrosurgical generator executing software that is configured to: measure a measured impedance value while provide a plurality of voltage pulses to a cutting instrument; and, partially or fully reduce a voltage amplitude of each of the plurality of voltage pulses when the measured impedance value exceeds an impedance threshold.

In some aspects, the techniques described herein relate to an RF electrosurgical system, wherein the impedance threshold is within an inclusive range of about 500 Ohms to about 2,000 Ohms.

In some aspects, the techniques described herein relate to an RF electrosurgical system, wherein the impedance threshold is a predetermined threshold value or a percentage impedance increase.

In some aspects, the techniques described herein relate to a RF electrosurgical system, wherein the impedance threshold is looked up in a database stored in memory of the RF electrosurgical generator based on a cutting instrument model number, serial number, or identification indicia.

In some aspects, the techniques described herein relate to a RF electrosurgical system, wherein the software is further configured to partially or fully reduce a voltage amplitude of each of the plurality of voltage pulses when a predetermined voltage amplitude threshold of one of the pulses of the plurality of pulses is exceeded and/or if a predetermined pulse time threshold of one of the pulses of the plurality of pulses is exceeded.

In some aspects, the techniques described herein relate to an RF electrosurgical system, wherein time intervals between the plurality of pulses are non-uniform.

In some aspects, the techniques described herein relate to an RF electrosurgical system, wherein the plurality of voltage pulses are arranged in bursts separated by relaxation periods.

In some aspects, the techniques described herein relate to an RF electrosurgical system, wherein the RF energy alternates within an inclusive range of about 4 MHz and 5 MHz

In some aspects, the techniques described herein relate to an RF electrosurgical system, wherein the RF energy alternates at 4.1, 4.2, 4.3, 4.4, 4.5. 4.6, 4.7, 4.8, 4.9, 5.0 MHz.

In some aspects, the techniques described herein relate to an RF electrosurgical system, wherein the RF energy is produced with a duty cycle of about 1% to about 20%.

In some aspects, the techniques described herein relate to a RF electrosurgical system, wherein the RF electrosurgical generator preventing the plurality of voltage pulses from occurring at one or more of atrial diastole, atrial systole, ventricular diastole, and/or ventricular systole.

In some aspects, the techniques described herein relate to an RF electrosurgical system, wherein the software is configured to monitor ECG data and limit the RF energy to only some, but not all, portions of a cardiac cycle of a patient.

In some aspects, the techniques described herein relate to an RF electrosurgical system, wherein the software displays a user interface configured to select which portions of a patient's cardiac cycle receives the RF energy.

In some aspects, the techniques described herein relate to an RF electrosurgical system, wherein the cutting instrument is a valve leaflet therapy device removal system.

In some aspects, the techniques described herein relate to an RF electrosurgical system, wherein the cutting instrument is a loop including one or a plurality of electrodes.

In some aspects, the techniques described herein relate to a RF electrosurgical system, wherein the cutting instrument is a cutting loop catheter including a plurality of electrodes that are each have separate electrical paths in communication with the RF electrosurgical generator.

In some aspects, the techniques described herein relate to a RF electrosurgical system, wherein the software is configured to monitor a measured temperature value from the cutting instrument and prevent the RF energy when the measured temperature value is above a predetermined temperature threshold.

In some aspects, the techniques described herein relate to an RF electrosurgical system, wherein the cutting instrument is an RF guidewire having a distal portion that forms a coil shape when unconstrained.

In some aspects, the techniques described herein relate to a RF electrosurgical system, wherein the software is configured to measure impedance from the cutting instrument, compare it to a tissue contact impedance threshold, and alert a user to tissue contact when the tissue contact impedance threshold is exceeded.

In some aspects, the techniques described herein relate to a RF electrosurgical system, wherein the software is configured to measure impedance from the cutting instrument, compare it to a device contact impedance threshold, and alert a user to tissue contact when the device contact impedance threshold is exceeded.

In some aspects, the techniques described herein relate to an RF electrosurgical system, wherein the software is configured to measure a time of use of the cutting instrument.

In some aspects, the techniques described herein relate to a method of operating a RF electrosurgical system, including: executing software on an RF electrosurgical generator including a user interface and one or more outlets supplying RF energy to an RF cutting instrument connected thereto; measure a measured impedance value with the RF electrosurgical generator while provide a plurality of voltage pulses to a cutting instrument; and, partially or fully reduce a voltage amplitude of each of the plurality of voltage pulses with the RF electrosurgical generator when the measured impedance value exceeds an impedance threshold.

In some aspects, the techniques described herein relate to an RF electrosurgical system, including: an RF electrosurgical generator including a user interface and one or more outlets supplying RF energy to an RF cutting instrument connected thereto; the RF electrosurgical generator executing software that is configured to: prevent the RF energy from being delivered to the cutting instrument at one or more of atrial diastole, atrial systole, ventricular diastole, and/or ventricular systole.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates a perspective view of an RF electrosurgical system 100, including an RF electrosurgical generator 102 that is configured to supply RF energy to one or more surgical instruments, according to some examples.

FIG. 2 is a graph of voltage amplitude versus time which illustrates example voltage train bursts 130 and relaxation periods 132, according to some examples.

FIG. 3 is a graph of voltage amplitude versus time which illustrates example voltage pulses 134 within each voltage train bursts 130, according to some examples.

FIG. 4 illustrates a graph of a voltage amplitude and a graph of an impedance of RF energy output, according to some examples.

FIG. 5 illustrates an ECG graph of voltage versus time that demonstrates a cardiac cycle of a patient, according to some examples.

FIG. 6 illustrates a side view of one example of a valve leaflet therapy device removal system 150 that may be used to remove a valve leaflet clip or similar device from a patient's heart, according to some examples.

FIG. 7 illustrates a view of a cutting loop 160 comprising only a single electrode 162 located at a distal end of the cutting loop 160 with adjacent insulated portions 164, according to some examples.

FIG. 8 illustrates a view of a cutting loop 166 having a plurality of electrodes 162A, 162B, 162C, according to some examples.

FIG. 9 illustrates a view of a cutting loop 166 having a plurality of electrodes 162A, 162B, 162C, according to some examples.

FIG. 10 illustrates distal portions of the capture basket catheter 152, snare catheter 154, and cutting loop catheter 156 that are placed underneath or in the ventricular side of a leaflet clip 40 that is connected to heart valve leaflets 20, according to some examples.

FIG. 11 illustrates the snare catheter 154 that may be positioned and tightened around the leaflet clip 40 and the leaflet clip 40 may be pulled towards or into the basket of the capture basket catheter 152, according to some examples.

FIG. 12 illustrates one example of a cutting loop device 170 having a hook, a “C” shape, or an open loop shape 172, according to some examples.

FIG. 13 illustrates a capture member 180 that may be an elongated wire, plurality of wires, tubular structure, or similar elongated structure, and may be positioned within the lumen of the catheter body 182, according to some examples.

FIG. 14 illustrates a capture member 180 that may be an elongated wire, plurality of wires, tubular structure, or similar elongated structure, and may be positioned within the lumen of the catheter body 182, according to some examples.

FIG. 15 illustrates another example of an RF guidewire 190 that may be advanced from a catheter or sheath 192, according to some examples.

FIG. 16 illustrates another example of an RF guidewire 190 that may be advanced from a catheter or sheath 192, according to some examples.

FIG. 17 illustrates a contact interface 200 displayable on the display 104 of the RF electrosurgical generator 102, according to some examples.

FIG. 18 illustrates an electrode activation interface 202 that allows a user to determine which electrodes will be electrically active when the RF power is delivered to the cutting instrument (e.g., cutting loop), according to some examples.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

The present specification is generally directed to various aspects of an improved RF electrosurgical system, including an RF electrosurgical generator. While the improved RF electrosurgical system may be used in a variety of different procedures, it may be particularly helpful for procedures involving or occurring within a heart of a patient.

In a first example, the RF electrosurgical system comprises an RF electrosurgical generator that supplies RF current to one or more electrosurgical instruments. In some instances, the RF electrosurgical generator may be configured to produce a cavitation “spark” that creates effective and repeatable wet field cutting while minimizing heat generation in the tissue. A cavitation spark is generally defined as the creation of a spark or plasma within a wet field environment between an active electrode and patient tissue, such that the cells of the tissue rapidly heat and explode before. If applied correctly, this cavitation spark may produce desirable cutting while limiting any excess heat to adjacent or nearby tissue. Additionally, such a cavitation spark may better allow for desirable cutting when the active electrode is fully enveloped in tissue, fully enveloped in flowing blood, or partially in contact with tissue and blood.

The RF electrosurgical generator creates such a cavitation spark with improved cutting by providing a plurality of pulses (e.g., a pulse train or burst) where each pulse is varied to provide a rapid voltage increase that results in high voltage peaks while controlling this voltage based on measured impedance. At a certain voltage, the electrical current of these voltage peaks may overcome the electrode-tissue interface and may produce cavitations sparks that locally disrupt the tissue and produces the desired cutting effect.

When a cavitation spark occurs, impedance will increase. Hence, impedance may be continually measured (or at least during application of voltage) and once it exceeds a predetermined threshold, a cavitation spark is presumed to be created. The voltage amplitude may be immediately reduced to thereby minimize excess heat generation. While the timing of the impedance increase appears relatively simultaneous with the cavitation spark caused by the high voltage, it is possible that the impedance may have a very small time offset (e.g., a several microseconds). However, for the purposes of generating one or more cavitations sparks for cutting purposes, such a small time offset may not significantly impact cutting performance.

This pulse process may be repeated, for example, after a brief relaxation period. These groups or bursts of pulses may be duty cycled to produce a timed average output power that is significantly lower than if it was a continuous signal (e.g., a duty cycle between 1% and 50%, or more specifically 10%).

Further details of these pulses are described later in this specification.

In another example, the RF electrosurgical generator may limit RF cutting energy to only certain portions of a patient's cardiac cycle. For example, cutting may be limited to either diastole or systole generally or one or more of atrial diastole, atrial systole, ventricular diastole, and/or ventricular systole. RF cutting energy may be limited to any combination of these cardiac phases (e.g., 1, 2, or 3 cardiac phases, as well as all cardiac phases). In a similar example, cutting may be limited to one or more portions of the ECG measurements, such as one or more portions of the P wave, the PR segment, the Q wave, the R wave, the S wave, the ST segment, the PR interval, the QRS complex, or the QT interval. Again, RF cutting energy may be limited to any combination of these ECG portions. This may be particularly helpful for procedures that involve cutting within a heart or in areas near or adjacent to a heart.

Many different procedures are possible with the RF energy limited to only portions of a patient's cardiac cycle. For example, a heart valve leaflet, such as a mitral valve, an aortic valve, a tricuspid valve, or a pulmonary valve, may be cut only at certain portions of a patient's cardiac cycle. In another example, heart valve therapies, such as a heart valve leaflet clip may be cut only at certain portions of a patient's cardiac cycle. In another example, RF energy may be limited when creating an opening through an atrial septum or a ventricular septum of a patient's heart.

The RF generator may limit RF energy to an active electrode by monitoring a heart of a patient. This monitoring may include monitoring an ECG reading of a patient. The RF generator may be configured to connect directly to ECG leads and thereby measure and record ECG data, or the RF generator may connect to a separate ECG machine and receive the ECG data from the ECG machine in real time during a procedure. The RF generator may include software stored in nontransitive memory and executed by a processor that analyzes the ECG data to determine a cardiac phase of a patient, compare the cardiac phase of the patient to a predetermined timing setting of RF energy (i.e., which portions of the ECG reading of a cardiac cycle RF energy should be applied to), and then supplying RF energy only at times in the cardiac cycle designated by the predetermined timing setting of RF energy.

In some examples, a cutting instrument may include an identification data such as a serial number, a device model number, and/or similar identification indicia. The identification data may be stored in readable memory within the cutting instrument (e.g., flash memory) and may be accessed by the RF electrosurgical generator when connected to the cutting instrument or via a wireless communication technique, such as via RFID or Wi-Fi. Additionally, the software of the RF electrosurgical generator may track the time of use of the cutting instrument, may sense contact with tissue or implant devices, or may control which electrodes RF energy may be sent to.

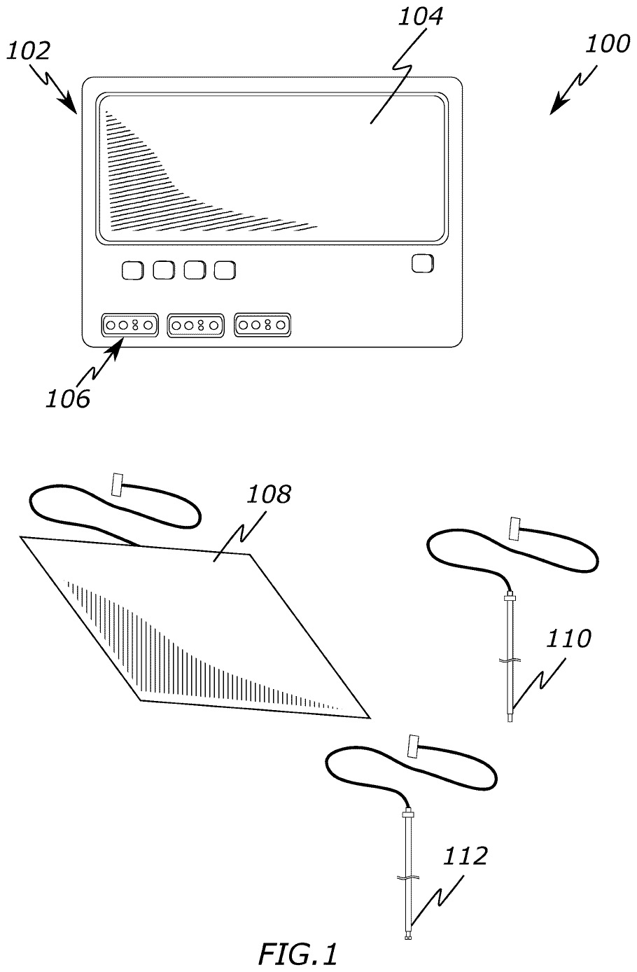

FIG. 1 illustrates a perspective view of an RF electrosurgical system 100, including an RF electrosurgical generator 102 that is configured to supply RF energy to one or more surgical instruments. As described in further detail below, the RF electrosurgical generator 102 includes several aspects that may improve cutting and/or piercing of tissue, particularly in wet field environments (e.g., environments containing bodily fluids such as blood, saline, or other fluids).

The RF electrosurgical generator 102 may take a variety of different forms but may generally comprise an outer housing supporting a display 104 and one or more outlets 106 that are configured to electrically connect to one or more surgical instruments, other equipment (e.g., an ECG measuring device), or other interface devices (keyboard, mouse, etc.).

In the present example, the display 104 may be a touch screen that allows user input to adjust or otherwise manipulate the software executed by the RF electrosurgical generator 102. Additionally or alternatively, other input interfaces may be included, such as physical buttons or a separately connected keyboard/mouse.

The RF electrosurgical generator 102 may be configured to connect to monopolar surgical instruments and/or bipolar surgical instruments. Typically, monopolar instruments include one “active” electrode that may be positioned within a patient to cut tissue and another electrode in external contact with a patient's skin. With regard to a monopolar example, FIG. 1 illustrates a pad electrode 108 that is positioned against a patient's skin and a monopolar catheter 110 that is positioned within the patient. Both the pad electrode 108 and monopolar catheter 110 are connected to the one or more outlets 106 of the RF electrosurgical generator 102 so that when and electrode on the monopolar catheter 110 contacts tissue within the patient, a circuit is completed and RF energy is applied at the location of the tissue contact. With regard to a bipolar example, FIG. 1 also illustrates a bipolar catheter 112 which includes two electrodes near its distal end that both have separate electrical paths back to the RF electrosurgical generator 102. When the two electrodes of the bipolar catheter 112 contact tissue, the circuit with the RF electrosurgical generator 102 is completed, thereby supplying RF energy to the contacted tissue.

The RF electrosurgical generator 102 may further include a processor configured to execute software code and non-transient memory configured to store software, data, and similar files. The processor may execute the software stored in the non-transient memory and display an interface on the display 104, as well as accept user input (e.g., from the touch screen aspect of the display 104) and accept data from any connected instruments, sensors, and/or devices (e.g., ECG machines).

The processor and software may further comprise a power supply comprising circuitry configured to supply RF energy through one or more of the one or more outlets 106 to one or more surgical instruments (e.g., pad electrode 108, monopolar catheter 110, and bipolar catheter 112).

For the purposes of this specification, RF energy (also referred to as RF current) is referred to as electrical current that alternates within a range of about 100 KHz to about 5.5 MHz. In some examples, the RF electrosurgical generator 102 may produce the previously described cavitation spark with RF energy within a range of about 4 MHz to about 5 Mhz. In a further example, the RF electrosurgical generator 102 may produce the previously described cavitation spark with RF energy at about 450 KHz.

The RF electrosurgical generator 102 may apply RF energy such that one or more surgical instruments create a cavitation spark between an active electrode and tissue in a wet field environment. As discussed in detail below, the RF electrosurgical generator 102 may apply the RF energy in such a way as to limit excess heat to adjacent nearby tissue, as well as improve cutting functionality, even when fully enveloped in tissue, fully enveloped in blood, or partially in contact with tissue and blood.

Several formulas are provided below to help better define the description of the RF energy output from the RF electrosurgical generator 102 that follows.

The output voltage of the RF electrosurgical generator 102 may be generally described by the following expression:

$$v ( t ) = A v ( t ) cos ( ω t )$$

Av(t) represents the time varying amplitude of the voltage.

ω is the radian frequency of the RF energy output and can also be expressed as ω=2πf, where f is the frequency in Hz of the output (e.g., but not limited to within a range of about 400-500 KHz, or about 450 KHz).

Output current can be measured via a suitable resistor, R, in series with the output current and measuring the voltage drop across it divided by the resistance. This can be represented by the expression:

$$i ( t ) = A i ( t ) cos ( ω t )$$

Ai(t) represents the time varying amplitude of the current (vr(t)/R).

ω is the radian frequency of the RF energy output as noted in the prior formula.

Since the resistor, R, can be a purely resistive (non-reactive) type, the phase angle between current and voltage will be zero so we may take the phase of the current to be the same as the phase of the voltage. θ is the phase angle between the output voltage and output current.

The load impedance can be computed from the measured output voltage and output current and may be described with the following formula:

Z(t)=v(t)/i(t)=|Za(t)|cos(ωt+θ); where θ is the phase angle between applied voltage, v(t) and the resulting current, i(t) and is dependent on the reactive component of the generator load formed by the interconnect cabling, electrode, electrode-tissue interface, tissue, ground return pad, and return cabling.

$$Where \vert Za ( t ) \vert = \vert Av ( t ) / Ai ( t ) \vert$$

As previously described, RF electrosurgical generator 102 produces an RF energy output that may demonstrate improved cutting between an active electrode and tissue of a patient. This improved cutting may be achieved by providing a plurality of pulses (e.g., a pulse train or burst) where each pulse is varied to provide a rapid voltage increase that results in voltage peaks while controlling or limiting this voltage based on measured impedance. At a certain voltage, the electrical current of these voltage peaks may overcome the electrode-tissue interface and may produce cavitations sparks that locally disrupt the tissue and produces the desired cutting effect.

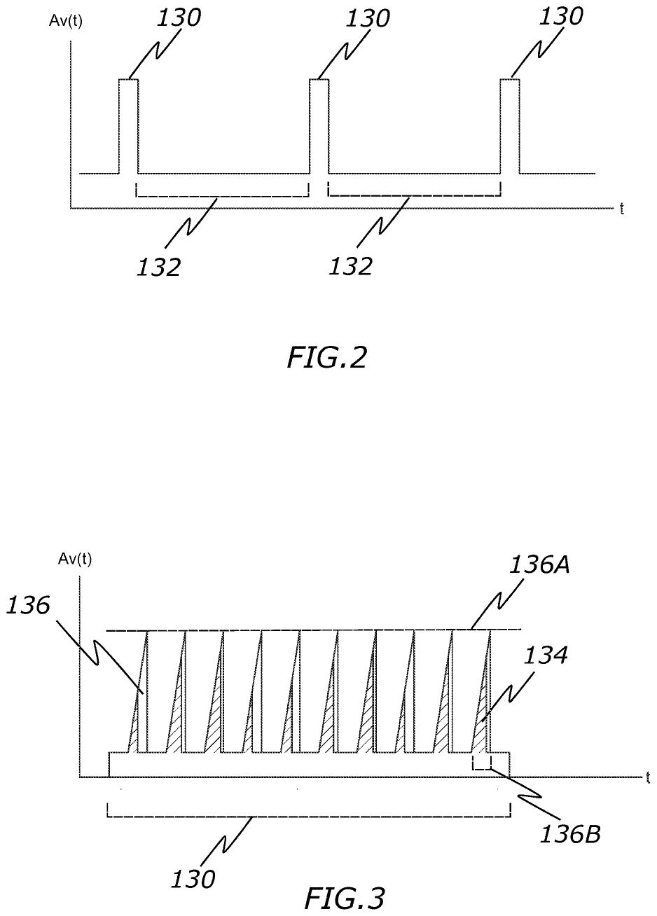

FIG. 2 is a graph of voltage amplitude versus time which illustrates example voltage train bursts 130 and relaxation periods 132 while FIG. 3 is a graph of voltage amplitude versus time which illustrates example voltage pulses 134 within each voltage train bursts 130.

The RF electrosurgical generator 102 may be configured to provide RF energy comprising a plurality of the voltage train bursts 130 with relaxation periods 132 between each voltage train bursts 130. Generally, the voltage train bursts 130, the voltage pulses 134, and the relaxation periods 132 are arranged such that they provide a time averaged output power that is significantly lower than if it was a continuous signal with no relaxation periods 132 or voltage pulses 134. For example, the RF electrosurgical generator 102 may configure this RF energy output to have a duty cycle within an inclusive range of about 0.1% to 50%, about 0.1% to 30%, about. 1% to about 20%, or about 0.1% to about 10%. In some specific examples, the duty cycle may be 1, 2, 3, 4, 5, 6, 7, 8, 9, 10, 11, 12, 13, 14, 15, 16, 17, 18, 19, or 20 percent.

The duty cycle may be calculated or estimated in several ways. In one example, the duty cycle may be the ratio between the time of a burst 130 and the total period of repetitive bursts 130. In this case, within each burst there is a time varying amplitude such that the total burst energy is less than the peak burst value times the burst duration. Further, each burst may have a different energy due to different voltage pulses, as described further below. Hence, it may be helpful to integrate the power delivered for the specific burst waveforms.

The RF electrosurgical generator 102 may be configured to provide RF energy having an alternating frequency within an inclusive range of about 100 KHz to about 5.5 MHz. In some examples, the RF energy may have a frequency within an inclusive range of about 4 MHz to about 5 MHz. Some specific examples include 4.1, 4.2, 4.3, 4.4, 4.5. 4.6, 4.7, 4.8, 4.9, 5.0 MHz, and frequencies in between. Other specific examples may include 4.41, 4.42, 4.43, 4.44, 4.45, 4.46, 4.47, 4.48, 4.49, 4.50, 4.51, 4.52, 4.53, 4.54, 4.55, 4.56, 4.57, 4.58, 4.59, 4.60 MHz, and frequencies in between. In general, 100 kHz tends to be the lower limit for this purpose to avoid the potential for generator current to stimulate nerves. Frequencies above 5 MHz may offer advantages in specific cases, but generally in the 4 MHz to 5 MHz range the reactance due to interconnect cables (both electrode and ground return pad cables) tends to be small so the load impedance appears relatively close to the real value (small reactance).

The relaxation periods 132 between voltage train bursts 130 may be the same or uniform in time between voltage train bursts 130 or may vary in time. Either option may help achieve a desired duty cycle for the overall RF energy output, however, varying the time between voltage train bursts 130 based on the voltage characteristics of each voltage pulses 134 (which may vary in amplitude and time) of a voltage train bursts 130 may be helpful to achieve a desired duty cycle and/or maintain a desired temperature of the active electrode or nearby tissue. Hence, in one example, the RF electrosurgical generator 102 may monitor voltage amplitudes for the plurality of voltage pulses 134 of a voltage train bursts 130, determine an average voltage of the voltage train bursts 130, compare the average voltage to a predetermined limit based on a duty cycle or temperature value at or near an active electrode, and then increasing or decreasing the relaxation periods 132 to achieve a desired duty cycle or temperature reading. In other examples, energy (power multiplied by time) or peak voltage may used instead of the average voltage.

In some examples, the relaxation periods 132 may be used to characterize ECG signals to better time voltage train bursts 130 with portions of the cardiac cycle, as discussed elsewhere in this specification. In some examples, the relaxation periods 132 may be adjusted to a desired cutting speed or tactile feel of the cutting such that shorter relaxation periods 132 may facilitate quicker cutting while longer relaxation periods 132 may cut slower and produce less heat. In that respect, the RF electrosurgical generator 102 may include a user interface that allows a user to increase or decrease the relaxation periods 132. In some examples, the relaxation periods 132 may be dynamically decreased following relatively high amplitudes of voltage train bursts 130 to help reduce or manage thermal delivery and/or to provide adequate time for the internal capacitors in the generator power supply to recharge and be ready for the next voltage train bursts 130.

As seen in FIG. 3, each of the voltage train bursts 130 includes a plurality of voltage pulses 134 (illustrated as a shaded area). The number of voltage pulses 134 may be a predetermined number or a variable number that depends on other calculations or measurements, such as duty cycle.

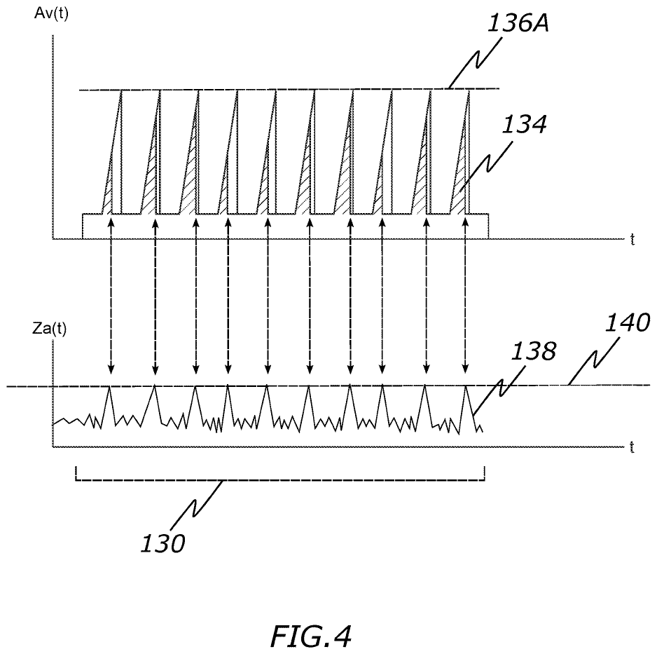

At the start of a voltage pulse 134, the voltage amplitude quickly increases over time. The voltage amplitude of a voltage pulse 134 may be partially or fully reduced (e.g., to zero voltage amplitude) based on a measured impedance value 138 of the RF energy, as seen in FIG. 4, which illustrates a graph of a voltage amplitude and a graph of an impedance of RF energy output. Specifically, the RF electrosurgical generator 102 may have an impedance threshold 140 at which a cavitation spark occurs or is likely to occur within a wet field environment. The impedance threshold 140 may be a predetermined threshold value, such as within an inclusive range of about 500 Ohms to about 2,000 Ohms. In some examples, the impedance threshold 140 may also be user adjustable. In some examples, the impedance threshold 140 may be dynamically calculated based on a percentage increase of the measured impedance value 138 or other algorithm (e.g., within a range of about 200% to about 10,000%).

In some examples, the impedance threshold 140 may be manufacturer calibrated based on the cutting instrument. The software of the RF electrosurgical generator 102 may store one or more threshold values associated with a specific cutting instrument and may further provide a user interface that allows the user to select the attached cutting instrument. Additionally or alternatively, the cutting instrument may include an identification (e.g., a model number, serial number, or identification indicia) stored within it (e.g., a memory chip or RFID chip) that the RF electrosurgical generator 102 may obtain to look up a suitable threshold value in a table or database file.

In some circumstances, the measured impedance value 138 may not elevate high enough to reach the impedance threshold 140. In such circumstances, other values may be used to trigger the voltage amplitude reduction. In one example, the voltage amplitude may be reduced (e.g., to zero) based on a predetermined voltage threshold 136A. In another example, the voltage amplitude may be reduced (e.g., to zero) based on a predetermined amount of time 136B. Further, both the predetermined voltage threshold 136A and predetermined amount of time 136B may be used to reduce the voltage amplitude (e.g., to zero). In this respect, FIGS. 3 and 4 illustrate an outline of the maximum voltage pulse 136.

In some examples, the time interval between voltage pulses 134 is a predetermined period of time. In other examples, each of the voltage pulses 134 are started at uniform starting times relative to the beginning of the voltage train bursts 130. In such an example, this may result in non-uniform intervals based on when the measured impedance value 138 reaches the impedance threshold 140.

In one example, the RF electrosurgical generator 102 may perform the following method, comprising increasing a voltage amplitude of RF energy supplied to one or more surgical instruments in a wet field environment, measuring a measured impedance value 138 of the RF energy (e.g., simultaneously with the voltage amplitude increase), and reducing the voltage amplitude (e.g., to zero) if 1) the measured impedance value 138 reaches an impedance threshold 140 and 2) if a predetermined voltage threshold 136A or predetermined time 136B is reached. After an amount of time (e.g., a predetermined amount of time), this cycle is started again to create a plurality of voltage pulses 134 as part of a voltage train burst 130.

This method or algorithm may produce many rapid voltage increases that will typically achieve cavitation sparks while maintaining a relatively lower duty cycle. Hence, desirable tissue cutting in a wet field environment is possible while maintaining a relatively low generation of excess heat (e.g., heat in adjacent tissue other than the tissue cells being cut).

The cutting instrument may also include a temperature sensor 163 at or near the electrode which may send temperature readings/data to the RF electrosurgical generator 102. The RF electrosurgical generator 102 may display this temperature reading on the display 104. The RF electrosurgical generator 102 may also include an audio and/or visual alarm that causes an alert when the measured temperature reading exceeds a predetermined threshold.

The RF electrosurgical generator 102 may also interrupt or adjust the RF energy output based on the predetermined temperature threshold. For example, the RF energy output may be completely stopped or the duty cycle may be changed (e.g., lowered).

The RF electrosurgical generator 102 may also be configured to deliver a fluid at timed intervals. In one example, dextrose or a similar fluid may be delivered near or over the one or more electrodes of a cutting instrument to enhance conductivity or other properties. This delivery of the fluid may be timed to occur only when power is delivered to the electrodes (e.g., during voltage train bursts 130).

The RF electrosurgical generator 102 may limit when RF energy output (e.g., voltage pulses 134) to an active electrode of a surgical instrument by monitoring a heart of a patient and applying the RF energy output to only certain portions of a patient's cardiac cycle that is less than an entire cardiac cycle. This monitoring may include monitoring an ECG reading of a patient. The RF electrosurgical generator 102 may be configured to connect directly to ECG leads and thereby measure and record ECG data (e.g., via one or more outlets 106), or the RF electrosurgical generator 102 may connect to a separate ECG machine and receive the ECG data from the ECG machine in real time during a procedure. The RF electrosurgical generator 102 may include software stored in nontransitive memory and executed by a processor that analyzes the ECG data to determine a cardiac phase of a patient, compare the cardiac phase of the patient to a predetermined timing setting of RF energy output (i.e., which portions of the ECG reading of a cardiac cycle RF energy should be applied to), and then supplying RF energy only at times in the cardiac cycle designated by the predetermined timing setting of RF energy.

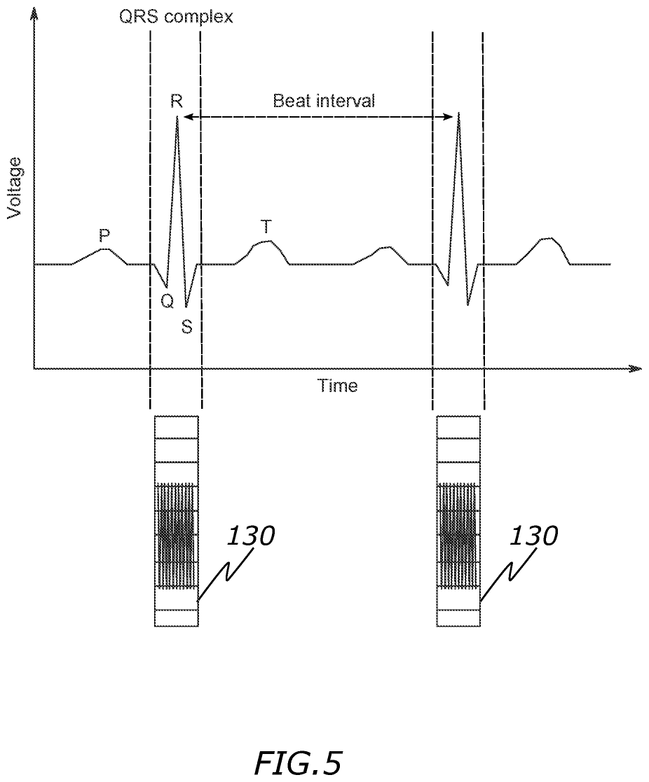

FIG. 5 illustrates an ECG graph of voltage versus time that demonstrates a cardiac cycle of a patient. In the present example, the voltage train bursts 130 are activated during the QRS complex of a patient's cardiac cycle, however any combination of measured ECG waves may be used, such as P, Q, R, S, T, and/or U, and segments therebetween. In another example, one or more of certain portions of the cardiac cycle may be avoided such as P, Q, R, S, T, and/or U, and segments therebetween.

The RF electrosurgical generator 102 may include a user interface that allows the user to select which portions of cardiac cycle may be activated for cutting, including the entire cardiac cycle.

Since different times of a cardiac cycle may result in different heart movements and amounts of blood moving through the heart, depending on the location of the active electrode of the cutting instrument, restricting cutting time of RF energy output to certain portions of the cardiac cycle of a patient may result in more reliable tissue cutting.

In one example, an anterior mitral valve leaflet may be cut to minimize risk of left-ventricular outflow tract obstruction. If the cutting instrument is held on the ventricular side of the mitral annular plane with the active electrode directed towards the anterior leaflet, as soon as the generator detects the start of systole (by looking for the ‘QRS complex’), it may deliver RF energy to the active electrode. Hence, the power delivery would be delivered with each heartbeat (minimizing total RF energy delivered).

In another example, the aortic valve leaflets may be cut to minimize risk of coronary obstruction by a TAVR implant, similar to cutting mitral leaflet tissue, except the operator may want to apply RF cutting energy in diastole only.

In another example removal of a heart valve leaflet clip (e.g., a Mitraclip) may need to be removed but may be fused to or in close proximity to chordae. If these chordae need to be cut, this cutting process can be a challenging as they are moving throughout the cardiac cycle. Hence, timing the delivery of RF energy to a time during the cardiac cycle when the chordae move in close to the active electrode may be advantageous to minimize RF energy delivery time and still cut the required tissue.

In another example, a slit may be cut in the interatrial septum for enabling larger bore catheters to cross without needing a balloon dilation. In trans-septal crossing procedures, there is a risk the operator can advance the needle too far into the left atrium while making the puncture. This can happen because the needle jumps or it requires higher than expected forward pressure to make the puncture. In the case of creating a puncture and/or slit for large bore catheter access, only applying RF energy and cutting tissue when the left atrium is at its largest size (early ventricular diastole), may help minimize the risk of inadvertent perforation or cutting outside the atrium.

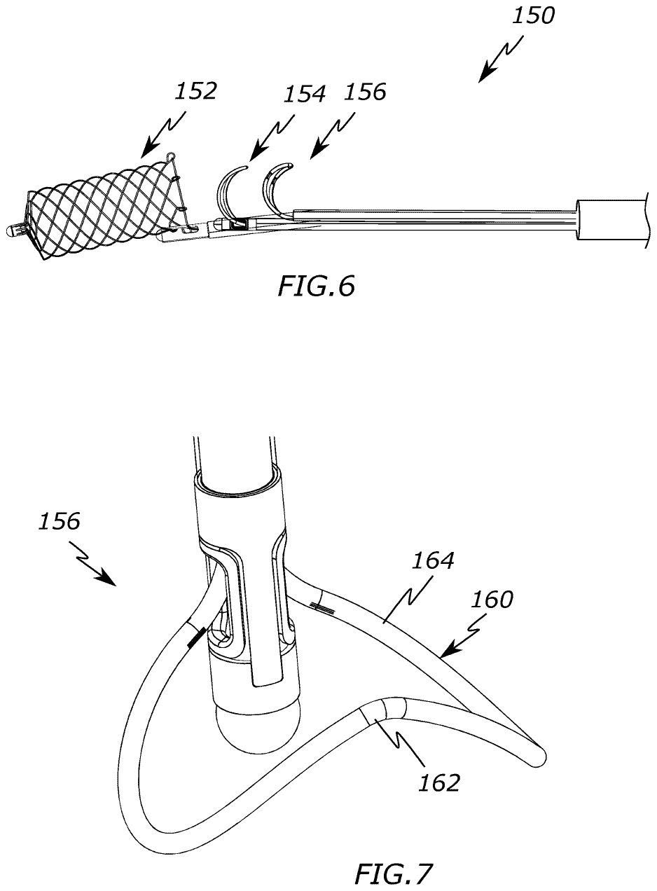

FIG. 6 illustrates a side view of one example of a valve leaflet therapy device removal system 150 that may be used to remove a valve leaflet clip or similar device from a patient's heart. Generally, the monopolar valve leaflet therapy device removal system 150 may comprise a capture basket catheter 152, a snare catheter 154, and a cutting loop catheter 156.

The capture basket catheter 152 may comprise an elongated catheter with a basket located at or near its distal end which is sized to encompass a heart valve therapy device. The snare catheter 154 may comprise an elongated catheter with a cinchable loop located at or near its distal end which is used to snare the heart valve therapy device. The cutting loop catheter 156 may comprise an elongated catheter with a cutting loop located at or near its distal end and which includes one or more electrodes. The proximal end may comprise an electrical cable that connects to the one or more outlets 106 of the RF electrosurgical generator 102 such that it can supply RF energy to the valve leaflet therapy device removal system 150. The valve leaflet therapy device removal system 150 may be monopolar (e.g., used with a pad electrode 108) or bipolar.

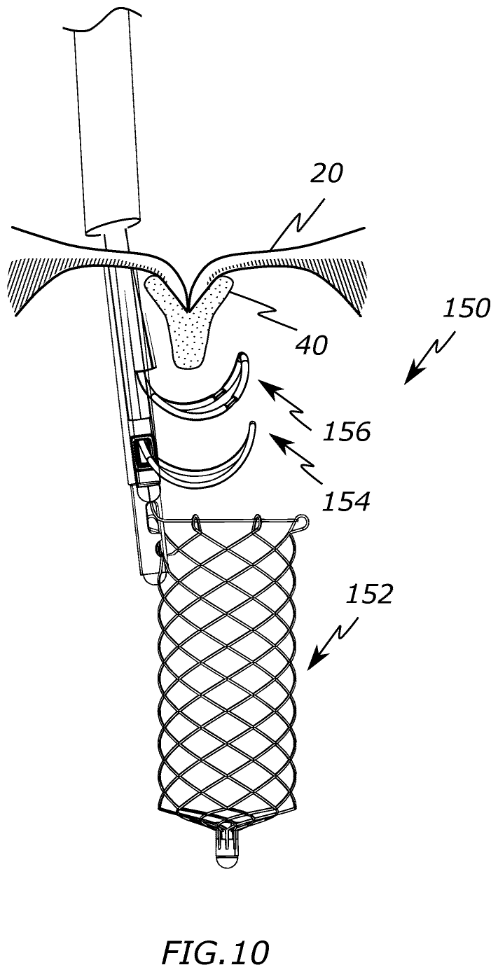

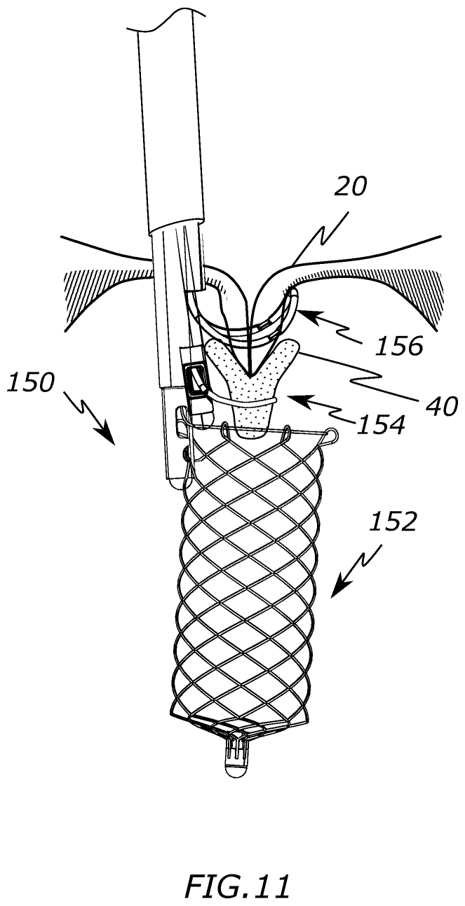

In FIG. 10, distal portions of the capture basket catheter 152, snare catheter 154, and cutting loop catheter 156 are placed underneath or in the ventricular side of a leaflet clip 40 that is connected to heart valve leaflets 20. In FIG. 11, the snare catheter 154 may be positioned and tightened around the leaflet clip 40 and the leaflet clip 40 may be pulled towards or into the basket of the capture basket catheter 152. The cutting loop of the 156 is positioned between the leaflet clip 40 and the heart valve leaflets 20. When the RF electrosurgical generator 102 is activated, the one or more electrodes on the cutting loop of the cutting loop catheter 156 cut the heart valve leaflets 20 and any other tissue needed to remove the leaflet clip 40, which is then pulled into the basket of the capture basket catheter 152. Additional details on this device, variations, and methods of use can be found in PCT/US2023/060773 which is hereby incorporated by reference.

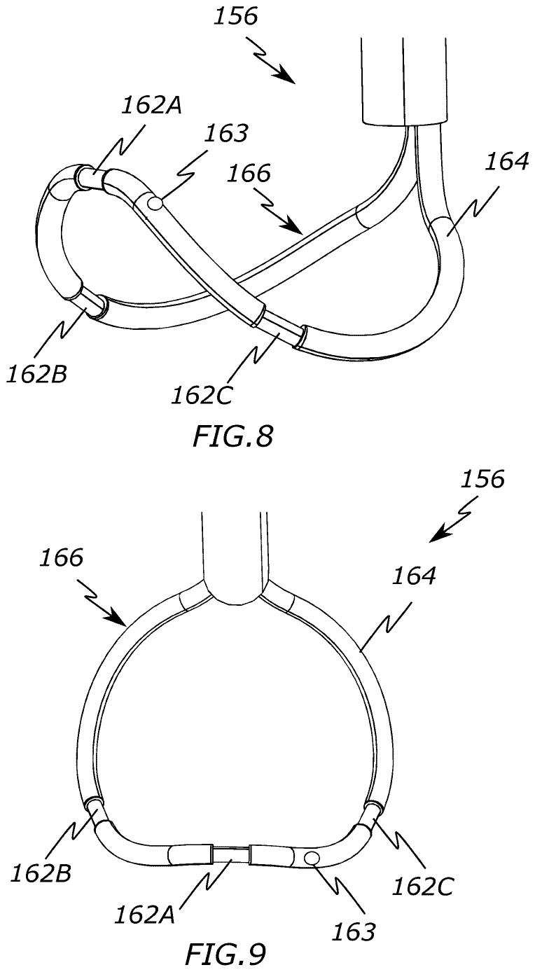

FIG. 7 illustrates a view of a cutting loop 160 comprising only a single electrode 162 located at a distal end of the cutting loop 160 with adjacent insulated portions 164. The cutting loop 160 may be used in a monopolar arrangement. FIGS. 8 and 9 illustrate views of a cutting loop 166 having a plurality of electrodes 162A, 162B, 162C. While three electrodes are illustrated, 2, 3, 4, 5, 6, 7, 8, or more electrodes are also possible. The electrodes may be positioned at various locations around the cutting loop 166, such as a middle location and at side locations. The cutting loop 160 may also include a temperature sensor which allows the RF electrosurgical generator 102 to monitor the temperature near the electrodes, as previously discussed in this specification.

FIG. 12 illustrates one example of a cutting loop device 170 having a hook, a “C” shape, or an open loop shape 172 at the end of an elongated shaft 174. The curved shape may lie substantially within a single plane or may spiral in a non-planar shape. The inner surface 172A may include one or more electrodes, the outer surface may include one or more electrodes, or electrodes may encompass both inner and outer surfaces. This shape may be particularly helpful for use in cutting chordae, in addition to tissue freeing a heart valve therapy device. Again, a proximal end or chord may be connected to the one or more outlets 106 of the RF electrosurgical generator 102 to create an electrical pathway to any electrodes on the open loop shape 172.

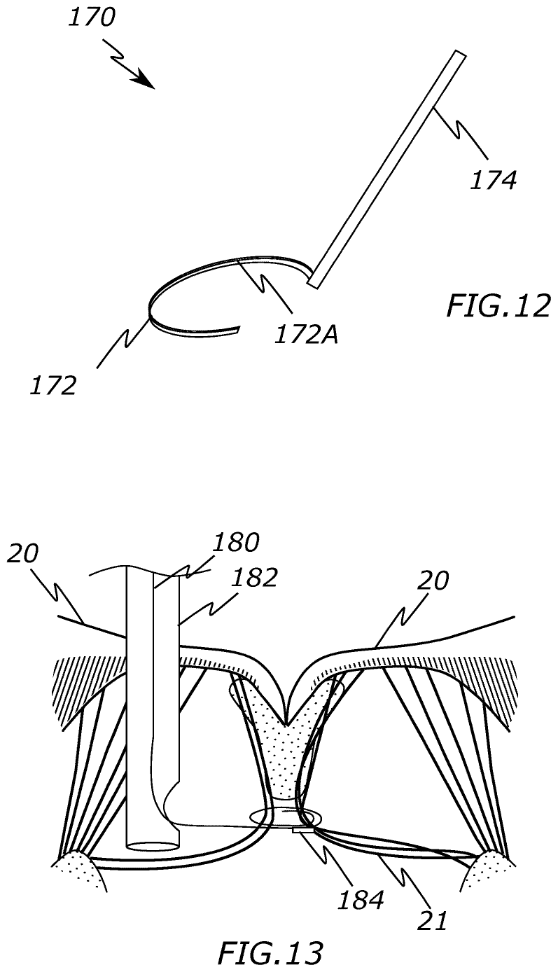

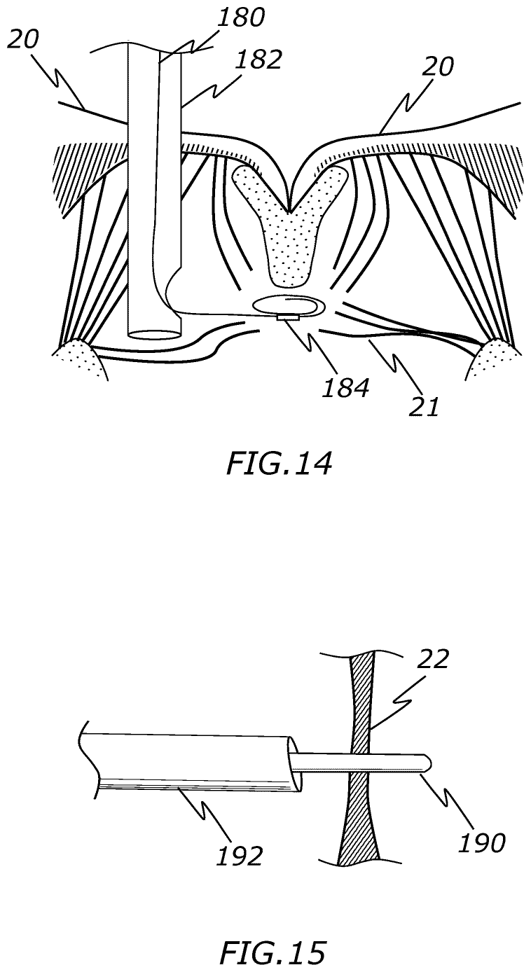

FIGS. 13 and 14 illustrate a capture member 180 that may be an elongated wire, plurality of wires, tubular structure, or similar elongated structure, and may be positioned within the lumen of the catheter body 182. In the present example, the distal end of the capture member 180 has a curved or spiral shape (e.g., either a spiral within a single plane or a helical/spiral shape) when unconstrained (memorized shape imparted to shape memory material). This memorized shape may be used to encircle one or more chordae 21. The capture member 180 may have at least a portion of its distal end uninsulated and in electrical communication with the RF electrosurgical generator 102 or one or a plurality of electrodes 184 may be included on a distal portion of the capture member 180.

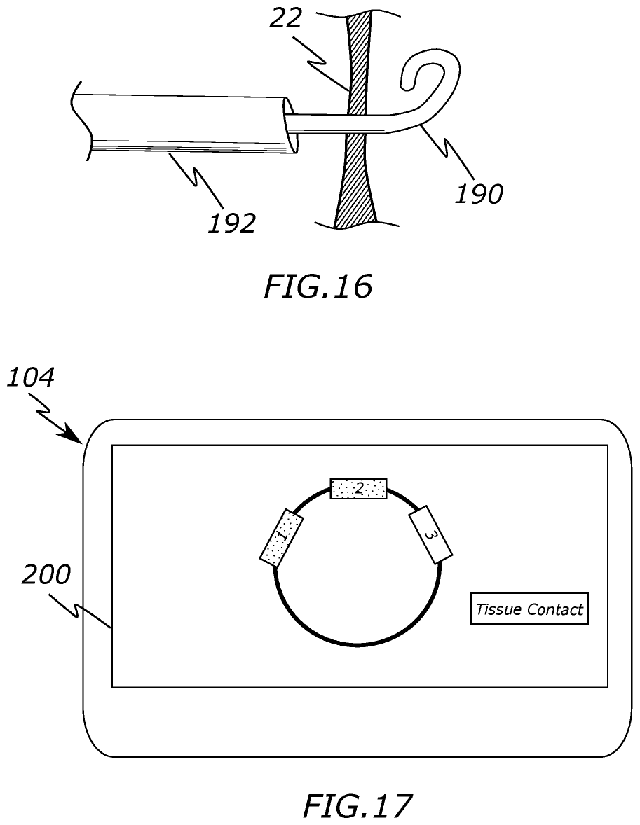

FIGS. 15 and 16 illustrate another example of an RF guidewire 190 that may be advanced from a catheter or sheath 192. The RF guidewire 190 may be composed of a shape memory material, such as Nitinol, and may have a distal portion with a memorized shape that curls, coils, or otherwise forms an atraumatic “pig tail” shape when unconstrained. The RF guidewire 190 may be in electrical communication with the RF electrosurgical generator 102, allowing RF energy to be delivered to its distal region. In the present example, the RF guidewire 190 may pass through a septum 22 of a heart for a transeptal crossing procedure.

The RF electrosurgical generator 102 may further include software that monitors impedance to determine if a heart valve therapy device, such as the previously discussed leaflet clip 40, is in contact with one or more of the electrodes of the instrument (e.g., cutting loop catheter 156). The RF electrosurgical generator 102 may compare a measured impedance value, compare the measured impedance value to a device contact impedance threshold, and then creating an audio and/or video alert (e.g., on the display 104).

The RF electrosurgical generator 102 may further include software that monitors impedance to determine tissue is in contact with one or more of the electrodes of the instrument (e.g., cutting loop catheter 156). The RF electrosurgical generator 102 may compare a measured impedance value, compare the measured impedance value to a tissue contact impedance threshold, and then creating an audio and/or video alert (e.g., on the display 104).

In the case of monitoring for the device contact impedance threshold and/or the tissue contact impedance threshold, if a cutting instrument includes a plurality of different electrodes on different circuits, the impedance of each electrode may be monitored separately and the RF electrosurgical generator 102 may display which of the electrodes are in contact with the device or tissue.

As previously discussed, the cutting instrument may include an identification (e.g., a model number or serial number) stored within it (e.g., a memory chip or RFID chip) that may be read by the RF electrosurgical generator 102. The software of the RF electrosurgical generator 102 may be configured to track the amount of time the cutting instrument is used. The software may also have a time of use threshold that, when exceeded, generates an audio and/or video alert indicating that a recommended lifetime use time has been exceeded. Hence, the software may store or track a time of use of a specific cutting element; compare the time of use of the cutting element to a predetermined time of use threshold (e.g., by looking up a threshold based on a manufacturer/model identification), and then generating an alert when the threshold has been exceeded. A similar threshold may additionally or alternatively be used for timing an amount of use time during a single procedure.

The software of the RF electrosurgical generator 102 may be further configured to monitor for and compensate for changing of impedance over its use during a procedure or the lifetime of a cutting instrument, such as due to the buildup of dead tissue on the electrodes (eschar). In one example, a small voltage may be applied to the active electrode during the relaxation periods 132 (small enough not to induce heat generation at the electrode) and measure the resulting current. This would allow the RF electrosurgical generator 102 to compute a “small signal” electrode impedance. The RF electrosurgical generator 102 may track this value over the procedure and infer information about the tissue buildup on the electrode. In another example, the RF electrosurgical generator 102 may may track impedance “peaks” occurring during or immediately following cutting events and infer this information.

As previously discussed, any of the cutting instruments disclosed in this specification may include a plurality of electrodes that are independently wired (i.e., that each form different circuits that can be independently actuated). For example, the cutting loop catheter 156 in FIGS. 8 and 9. Such a multi-electrode cutting loop may be connected to the RF electrosurgical generator 102 that can independently sense and energize these electrodes, allowing a user to determine if a specific electrode is in contact with tissue or blood, and then specify which of the electrodes should be electrically activated.

For example, FIG. 17 illustrates a contact interface 200 displayable on the display 104 of the RF electrosurgical generator 102. The contact interface 200 may display information indicating which of the electrode plurality of electrodes is in contact with tissue and/or an implant device (e.g., electrodes 1, 2, and/or 3). The tissue contact display may be in the form of a graphical depiction of a cutting loop as seen in FIG. 17, numerical indications, or variations thereof. Tissue contact may be determined by continuously measuring electrical values for each electrode, such as impedance, and when the electrical value exceeds a certain threshold, tissue contact would be determined.

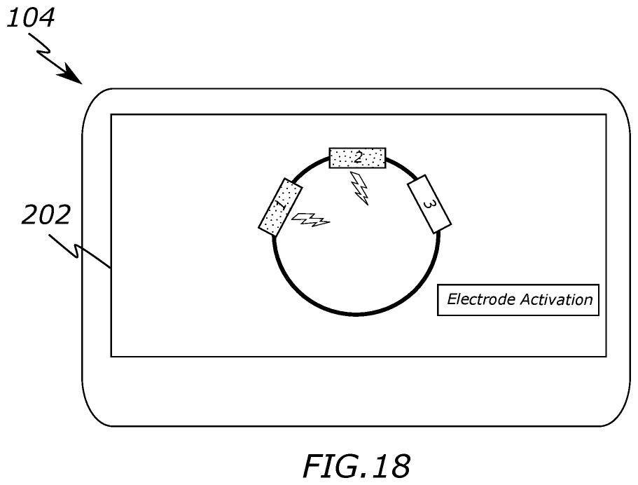

FIG. 18 illustrates an electrode activation interface 202 that allows a user to determine which electrodes will be electrically active when the RF power is delivered to the cutting instrument (e.g., cutting loop). The electrode activation interface 202 may be in the form of a graphical depiction of a cutting loop as seen in FIG. 18, numerical indications, or variations thereof. They display may also include indicia, symbols, words, or other display elements that indicate if an electrode is active or deactivated. Additionally, the activation display and the tissue contact display may be incorporated into the same screen so as to show data at the same time. The software of the RF electrosurgical generator 102 may first display the electrode activation interface 202, then the user may select the electrodes to be activated, information corresponding to the selected electrodes is stored in memory, and then the software supplies RF energy to only the selected electrodes of the cutting instrument.

It should be emphasized that any of the examples discussed in this application may be mixed and matched together. While some examples are separately described from others, any combination of these features is specifically contemplated.

1-23. (canceled)

24. An RF electrosurgical system, comprising:
an RF electrosurgical generator comprising a user interface and one or more outlets supplying RF energy to an RF cutting instrument connected thereto; the RF electrosurgical generator executing software that is configured to:
measure a measured impedance value while providing a plurality of voltage pulses to a cutting instrument; and,
increase a voltage amplitude of each of the plurality of voltage pulses until the measured impedance value of each pulse exceeds an impedance threshold and then partially or fully reduce the voltage amplitude.

25. The RF electrosurgical system of claim 1, wherein the impedance threshold is associated with creation of a cavitation spark.

26. The RF electrosurgical system of claim 1, wherein the impedance threshold is within an inclusive range of about 500 Ohms to about 2,000 Ohms.

27. The RF electrosurgical system of claim 1, wherein the impedance threshold is a predetermined threshold value or a percentage impedance increase.

28. The RF electrosurgical system of claim 1, wherein the impedance threshold is looked up in a database stored in memory of the RF electrosurgical generator based on a cutting instrument model number, serial number, or identification indicia.

29. The RF electrosurgical system of claim 1, wherein the software is further configured to partially or fully reduce a voltage amplitude of each of the plurality of voltage pulses when a predetermined voltage amplitude threshold of one of the pulses of the plurality of pulses is exceeded and/or if a predetermined pulse time threshold of one of the pulses of the plurality of pulses is exceeded.

30. The RF electrosurgical system of claim 1, wherein time intervals between the plurality of pulses are non-uniform.

31. The RF electrosurgical system of claim 1, wherein the plurality of voltage pulses is arranged in bursts separated by relaxation periods.

32. The RF electrosurgical system of claim 1, wherein the RF energy alternates at about 100 kHz or higher.

33. The RF electrosurgical system of claim 1, wherein the RF energy alternates at 410, 420, 430, 440, 450, 460, 470, 480, 490, or 500 KHz.

34. The RF electrosurgical system of claim 1, wherein the RF energy is produced with a duty cycle of about 1% to about 20%.

35. The RF electrosurgical system of claim 1, wherein the RF electrosurgical generator prevents the plurality of voltage pulses from occurring at one or more of atrial diastole, atrial systole, ventricular diastole, and/or ventricular systole.

36. The RF electrosurgical system of claim 1, wherein the software is configured to monitor ECG data and limit the RF energy to only some, but not all, portions of a cardiac cycle of a patient.

37. The RF electrosurgical system of claim 1, wherein the software displays a user interface configured to select which portions of a patient's cardiac cycle receives the RF energy.

38. The RF electrosurgical system of claim 1, wherein the software is configured to measure impedance from the cutting instrument, compare it to a tissue contact impedance threshold, and alert a user to tissue contact when the tissue contact impedance threshold is exceeded.

39. The RF electrosurgical system of claim 1, wherein the software is configured to measure impedance from the cutting instrument, compare it to a device contact impedance threshold, and alert a user to tissue contact when the device contact impedance threshold is exceeded.

40. The RF electrosurgical system of claim 1, wherein the software is configured to measure a time of use of the cutting instrument.
